(19) United States
(12) Patent Application Publication
(10) Pub. No.: US 2015/0031952 A1
(43) Pub. Date: Jan. 29, 2015

(54) Flexible/Rigid sheath guiding mechanism and integrated all-around adjustable flexible/rigid endoscope thereof

(71) Applicant: Gang LONG, Hubei (CN)

(72) Inventors: Gang Long, Wuhan (CN); Xueping Long, Wuhan (CN)

(21) Appl. No.: 14/355,379

(22) PCT Filed: Oct. 31, 2012
(86) PCT No.: PCT/CN2012/083798

§ 371 Date: Aug. 26, 2014

(30) Foreign Application Priority Data
Nov. 1, 2011 (CN) 201120425682.2
Nov. 2, 2011 (CN) 201110341167.0
Nov. 2, 2011 (CN) 201120427977.3
Nov. 3, 2011 (CN) 201120429968.8
Nov. 7, 2011 (CN) 201110349536.0
Nov. 7, 2011 (CN) 201120437883.4
Nov. 7, 2011 (CN) 201120437992.6
Nov. 8, 2011 (CN) 201110349641.4
Nov. 8, 2011 (CN) 201120438043.X

(51) Int. Cl. A61B 1/00; A61B 1/01

(52) U.S. Cl. CPC A61B 1/00154; A61B 1/00135; A61B 1/01
USPC 600/114

(57) ABSTRACT

A flexible or rigid sheath guiding mechanism, comprises: a splitter (101), a guiding sleeve tube (102), a flexible sheath (103), a rigid sheath (104) and a handle (105). A first end of the guiding sleeve tube (102) is mounted on an outlet of the splitter (101), the guiding sleeve tube (102) and provided coaxially with the outlet of the splitter (101). A second end of the guiding sleeve tube is inserted in the rigid sheath (104). The guiding sleeve tube (102) fits the rigid sheath (104). The handle (105) comprises a handle head (106) and a handle sleeve (107) which are fitted with each other. The splitter (101) is mounted in the handle head (106). The rigid sheath (104) is mounted on the handle sleeve (107). The flexible sheath (103) goes through the guiding sleeve tube (102) and the rigid sheath (104), and an end of the flexible sheath (103) is mounted on the outlet of the splitter (101). The flexible or rigid sheath guiding mechanism and an integrated all-around adjustable flexible-rigid endoscope thereof have characteristics of precise guiding, reliable positioning, simple structures, and convenient assembly.

### CROSS REFERENCE OF RELATED APPLICATION

This is a U.S. National Stage under 35 U.S.C 371 of the International Application PCT-CN2012-083798, filed Oct. 31, 2012, which claims priority under 35 U.S.C. 119(a-d) to 201120425682.2 (CN), filed 1 Nov. 2011; 201110341167.0 (CN), filed 2 Nov. 2011; 201120427977.3 (CN), filed 2 Nov. 2011; 201120429968.8 (CN), filed 3 Nov. 2011; 201110349536.0 (CN), filed 7 Nov. 2011; 201120437883.4 (CN), filed 7 Nov. 2011; 201110349580.1 (CN), filed 7 Nov. 2011; 201120437992.6 (CN), filed 7 Nov. 2011; 201110349641.4 (CN), filed 8 Nov. 2011; and 201120438043.X (CN), filed 8 Nov. 2011.

### BACKGROUND OF THE PRESENT INVENTION

1. Field of Invention

The present invention relates to a medical device, and more particularly to a flexible-rigid sheath guiding mechanism and an integrated all-around adjustable flexible-rigid endoscope therefor.

2. Description of Related Arts

In clinical medicine, adopting the endoscope is currently the most commonly used diagnosis and treatment method with small damages on patients. Utilizing the conventional endoscope for treatment requires cooperation of a plurality of doctors, and the diagnosis and treatment effect thereof is highly depends on skill and experience of doctors. Since the endoscope stays a long time in the body of the patient for inspection, which requires the physicians to have excellent skills, and requires the endoscope to have high stability and sensitivity. In order to avoid repeatedly angle adjusting and positioning in the body of patients, the endoscope must have high security and high efficiency for meeting urgent requirements of clinical diagnosis and treatment. When the endoscope is intervened in the human body, a great extrusion force may be generated between the lens of the endoscope and the human tissue due to mis-operation caused by fatigue after a long time operation. When the extrusion force exceeds a certain threshold value, the soft tissue will be damaged, which further causes medical accidents such as perforation of human tissue and avulsion of natural orifice. Nowadays, the conventional endoscopes are classified in two types, wherein the first type thereof utilizing an angle sliding block for limitation, the second type thereof is a traction device with a locking mechanism. The first type is simple in structure, but the angle limitation lacks reliability. The second type has good independence and reliability in limitation, but the structure thereof is complex, and requires high relevance and interactive in the assembly of each part. The second type of endoscope is highly dependent on other devices of the endoscope, and requires the whole endoscope to adjust for coordination, and thus the utilization thereof is low.

Before this application, a Chinese patent application with a publication number of CN201010266551.4 discloses a modular flexible-rigid endoscope, in which by utilizing relative movement between the handle sleeve and the handle body cover of the switching handle, relative expand or contract between the external tube and the internal tube is achieved. In the patent, the tube comprises a rigid tube and a flexible sheath which are made of stainless steel and plastic, wherein the rigid tube is sleeved outside the flexible sheath. The rigid tube is in a fix thread connection with the handle sleeve by via a connecting flange. The flexible sheath passes a through hole in a center of the handle sleeve, so as to be fixedly connected with a port of the connector.

During the course of a surgical operation, the flexible sheath is directly contacted with the rigid sheath in the endoscope, and processed relative motion. Since the manufacture material of the flexible sheath is soft and deformable, the flexible sheath is easy to bend during the process of replacing the devices in the flexible sheath continuously, so that after a long-term utilization, the sensitivity of the devices is decreased, the loss of the endoscope increases, and the fiber scope or the image and lighting sensor is easily to be damaged. Furthermore, the bending of the tube causes inconvenient to the operation, which brings security threats to the operation. In addition, the mechanism for achieving the relative expand-contract and rotation between the external tube and the internal tube of the endoscope is not perfect and operation thereof is not convenient.

### SUMMARY OF THE PRESENT INVENTION

In order to overcome shortcomings illustrated in the conventional arts, an object of the present invention is to provide a flexible-rigid sheath guiding mechanism and an integrated all-around adjustable flexible-rigid endoscope therefore, which has characteristics of easy operation, high accuracy in repeat operation, no deformation and high stability.

Accordingly, in order to accomplish the above objects, the present invention provides a flexible-rigid sheath guiding mechanism for an endoscope, comprising: a splitter, a guiding sleeve tube, a flexible sheath, a rigid sheath and a handle, wherein a first end of the guiding sleeve tube is mounted on an outlet of the splitter, the guiding sleeve tube and provided coaxially with the outlet of the splitter 101, a second end of the guiding sleeve tube is inserted in the rigid sheath, the guiding sleeve tube fits the rigid sheath, the handle comprises a handle head and a handle sleeve which are fitted with each other, the splitter is mounted in the handle head, the rigid sheath is mounted on the handle sleeve, the flexible sheath goes through the guiding sleeve tube 102 and the rigid sheath, and an end of the flexible sheath is mounted on the outlet of the splitter.

Preferably, the splitter comprises a fixing part, a main channel for inserting the guiding sleeve tube and the flexible sheath is provided on an outlet of the fixing part, four distribution channels opposite to the main channel are provided on the fixing part, wherein outer ends of the four distribution channels extends to an end face of the fixing part, inner ends of the four distribution channels extend to gather in an inner end of the main channel.

Preferably, each central axis of the four distribution channels forms an included angle of 7°˜13° with an central axis of the fixing part.

Preferably, a fastening screw is provided on a side face of the main channel of the splitter, so as to fix the flexible sheath provided on the main channel of the splitter.

Preferably, the guiding sleeve tube is made of stainless steel, and welded together with the main channel on the outlet of the splitter.

Preferably, the flexible sheath has three entercloses running along an axis provided therein.

Preferably, both the handle head and the handle sleeve are cylindrical structures and are matched to sleeve together.

Preferably, a front end of the rigid sheath (104) is connected with the handle sleeve via a spring cartridge.

Preferably, in the technical solution mentioned above, the flexible-rigid sheath guiding mechanism and integrated all-around adjustable flexible-rigid endoscope thereof, further comprises a bending angle adjusting mechanism for the flexible sheath of the endoscope, a steering and positioning mechanism provided between the flexible sheath and the rigid sheath of the endoscope, and a flexible sheath pushing mechanism provided opposite to the rigid sheath for the flexible sheath of the endoscope.

Preferably, the angle adjusting mechanism comprises an angle adjusting steel wire, a handle, a nut and a screw, wherein the screw is provided on a location hole of the handle, the nut is provided on a first limit groove of the handle, so as to cooperate with the screw, wherein an end of the first limit groove extends to a surface of the handle and has an opening, a part of an external surface of the nut extends to the opening of the first limit groove on the handle, and the angle adjusting steel wire is connected with the screw.

Preferably, the screw is parallel to an axis of the handle.

Preferably, the screw is connected with the angle adjusting steel wire via a connector, the connector is a in a shape of a column and has screw threads on a first end thereof, a screw hole is provided on the screw, the connector 207 is connected with thread of the screw, a connecting hole is provided on a second end of the connector, and the angle adjusting steel wire is mounted on the connecting hole.

Preferably, the location holes are respectively provided on two axially opposite sides of the first limit groove 203.

Preferably, a bearing is provided between the location hole 205 and the screw 206.

Preferably, a length of the first limit groove is adaptive to a length of the nut.

Preferably, the steering and positioning mechanism comprises a handle head, a handle sleeve, an eccenter, a driving lever and a spindle, a location hole for receiving the spindle is provided in the handle head, a first end of the spindle is connected with the eccenter, a second end thereof is connected with the driving lever, wherein the spindle is provided along an axial direction of the handle head, an open groove is provided along a circumferential direction of a surface of the handle head, the driving lever is provided in the open groove, a bottom of the driving lever is fixedly connected with the spindle, and the eccenter is eccentrically provided on the spindle 305.

Preferably, a side face of an end of the open groove has a second limit groove, when the driving lever is received by the second limit groove, the eccenter is compressed on an inner surface of the handle sleeve, and in a locked position.

Preferably, a surface of the eccenter is in a circular arc shape.

Preferably, the flexible sheath pushing mechanism comprises the flexible sheath, a flexible sheath cap and the rigid sheath, wherein the flexible sheath cap is in a shape of a circular tube, an external surface of the flexible sheath cap has two stepped faces with different diameters, wherein an external surface of a first stepped face with a greater diameter is in a streamlined shape along the axial direction, an external surface of a second stepped face with a smaller diameter is a circular surface which is linear along the axial direction and capable of matching with an inner bore of the rigid sheath, an end face with a greater diameter of the flexible sheath cap 402 is an inclined plane, and a barrier chip for fixing steel wire is provided on the end face, an inner bore of the flexible sheath cap is sleeved on an external surface of a head of the flexible sheath, and is mounted thereon, an end of the steel wire in the flexible sheath 103 is fixedly connected with the barrier chip; the rigid sheath 104 is sleeved on an external of the flexible sheath, an external end face of the rigid sheath is capable of matching with a stepped end face of the flexible sheath cap.

Preferably, a step height h between the first stepped face and the second step face of the flexible sheath cap is equal to a thickness of the rigid sheath matched therewith.

Preferably, the barrier chip and the flexible sheath cap forms an integrated structure, the barrier chip is provided on a farthest portion of the inclined end face.

Preferably, the barrier chip is provided on a position corresponding to a steering control channel on a cross section of the flexible sheath cap 402.

In order to achieve the objects mentioned above, the present invention further provides a flexible-rigid sheath guiding mechanism for an endoscope, comprising: a splitter, a guiding sleeve tube, a flexible sheath, a rigid sheath and a handle, wherein a first end of the guiding sleeve tube is mounted on a main channel of the splitter, the guiding sleeve tube and provided coaxially with the main channel of the splitter 101, a second end of the guiding sleeve tube is inserted in the rigid sheath, the guiding sleeve tube fits the rigid sheath, the splitter is mounted on the handle, the flexible sheath goes through the guiding sleeve tube and the rigid sheath, and an end of the flexible sheath is mounted on the main channel of the splitter, an external surface of the rigid sheath is fixed and has an annular ligament for holding.

Preferably, the splitter is a part of a head of the handle, a circular main channel is provided on a front end of the splitter, wherein the guiding sleeve tube is provided on the main channel, a gap is provided between an external surface of the guiding sleeve tube and an internal surface of the main channel, a rear end of the rigid sheath is sleeved on an external of the guiding sleeve tube and is inserted into the gap, three distribution channels are provided on an end opposite to the main channel on the splitter, and inner ends of the three distribution channels extend to be gathered in an inner end of the main channel.

Preferably, the three distribution channels respectively comprise a apparatus catheter, a stainless steel capillary, and an imaging and lighting sensor catheter, wherein a chamber is provided on a root of the splitter, internal ends of the apparatus catheter, the stainless steel capillary, the imaging and lighting sensor catheter, the guiding sleeve tube and the flexible sheath extend to gather in the internal end of the main channel.

Preferably, the each central axis of the three distribution channels forms an included angle of 7°˜13° with an central axis of the main channel.

Preferably, the flexible sheath has three entercloses running along an axis provided therein, the three entercloses are respectively communicated with the three distribution channels of the splitter.

Preferably, the flexible-rigid sheath guiding mechanism for the endoscope, further comprises: a bending angle adjusting mechanism for the flexible sheath of the endoscope, a steering and positioning mechanism provided between the flexible sheath and the rigid sheath of the endoscope, and a flexible sheath pushing mechanism provided opposite to the rigid sheath for the flexible sheath of the endoscope.

Preferably, the steering and positioning mechanism comprises a collet which is matching with thread on a front end of the main channel of the splitter, the collet provided on the front end of the main channel is capable of processing elastic contraction, when the collet is tightened, the front end of the main channel is contracted to grip the rigid sheath, in such a manner that the rigid sheath is fixedly connected with the splitter.

Preferably, the angle adjusting mechanism comprises an angle adjusting steel wire, a handle, a nut and a screw, wherein the screw is provided on a location hole of the handle, the nut is provided on a first limit groove of the handle, so as to cooperate with the screw, wherein an end of the first limit groove extends to a surface of the handle and has an opening, a part of an external surface of the nut extends to the opening of the first limit groove on the handle, and the angle adjusting steel wire is connected with the screw.

Preferably, the screw is parallel to an axis of the handle.

Preferably, the location holes are respectively provided on two axially opposite sides of the first limit groove 203.

Preferably, a bearing is provided between the location hole and the screw.

Preferably, a length of the first limit groove is adaptive to a length of the nut.

Preferably, the flexible sheath pushing mechanism comprises the flexible sheath, a flexible sheath cap and the rigid sheath, wherein the flexible sheath cap is in a shape of a circular tube, an external surface of the flexible sheath cap has two stepped faces with different diameters, wherein an external surface of a first stepped face with a greater diameter is in a streamlined shape along the axial direction, an external surface of a second stepped face with a smaller diameter is a circular surface which is linear along the axial direction and capable of matching with an inner bore of the rigid sheath, an end face with a greater diameter of the flexible sheath cap 402 is an inclined plane, and a barrier chip for fixing steel wire is provided on the end face, an inner bore of the flexible sheath cap is sleeved on an external surface of a head of the flexible sheath, and is mounted thereon, an end of the steel wire in the flexible sheath 103 is fixedly connected with the barrier chip; the rigid sheath 104 is sleeved on an external of the flexible sheath, an external end face of the rigid sheath is capable of matching with a stepped end face of the flexible sheath cap.

Preferably, a step height h between the first stepped face and the second step face of the flexible sheath cap is equal to a thickness of the rigid sheath matched therewith.

Preferably, the barrier chip and the flexible sheath cap forms an integrated structure, the barrier chip is provided on a farthest portion of the inclined end face.

Preferably, the barrier chip is provided on a position corresponding to a steering control channel on a cross section of the flexible sheath cap 402.

In the integrated all-around adjustable flexible-rigid endoscope according to preferred embodiment of the present invention, the guiding sleeve tube adopts a rigid material, which is preferably a stainless steel. The guiding sleeve tube is finally welded to the splitter in the endoscope. The guiding sleeve tube made of stainless steel has effects of fixing, guiding and positioning. In the surgical operation, the steering steel is pulled to retract, so as to pull the whole flexible sheath to turn. After release, due to the steel characteristic of the guiding steel tube, the whole flexible sheath is capable of returning rapidly under the restriction of the guiding sleeve tube. Compared with the prior art, the integrated all-around adjustable flexible-rigid endoscope of the present invention is not only simple in structure, but also capable of forming an effective connection between the internal flexible sheath and the external rigid sheath, in such a manner that turning, expanding and contracting of the flexible sheath are all restricted by the stainless steel guiding sleeve tube in the rigid sheath guiding mechanism, in such a manner that the internal flexible sheath is fixed in a pathway, and that the flexible sheath can not retract, turn around or deform, and is capable of achieving repeated operation, so as to bring safeguard to the security of the operation. The clinical significance of the present invention lies in being capable of solving technical problems such as bending, deforming and poor restorability caused by repeated operation of the flexible sheath in the surgical procedures. The integrated all-around adjustable flexible-rigid endoscope of the present invention is safe and sensitive in clinical utilization.

The angle adjusting mechanism of the present invention has characteristics of reliable in limiting, simple in structure, simple in processing and assembling, highly dependent, and convenient in adjusting and fixing.

The flexible sheath pushing mechanism of the present invention is simple in structure and convenient for manufacture, the outer surface of the flexible sheath cap is designed to be smooth, in such a manner that in the repeated operation of the endoscope, the contacting surface with human tissue is not sharp but smooth, in such a manner that the human tissue is not damaged due to oversize puncture power, so as to provide protection on the tissue of the patients to some extent. When the flexible sheath and the rigid sheath of the endoscope are utilized cooperatively, the flexible sheath cap has an effect of fixing connections between the flexible sheath and the rigid sheath, in such a manner that during the operation, with turning around and bending of the flexible sheath, the flexible sheath can not retract to the rigid sheath tube, in such a manner that the operation thereof is more convenient, and that the adaptability degree of the flexible sheath and the rigid sheath is more high. In summary, the flexible or rigid sheath guiding mechanism and an integrated all-around adjustable flexible-rigid endoscope thereof have characteristics of precise guiding, reliable positioning, simple structures, and convenient assembly.

These and other objectives, features, and advantages of the present invention will become apparent from the following detailed description, the accompanying drawings, and the appended claims.

### BRIEF DESCRIPTION OF THE DRAWINGS

FIG. 1 is a structural sketch view of a flexible-rigid sheath guiding mechanism according to a preferred embodiment 1 of the present invention.

FIG. 2 is a structural sketch view of a splitter in FIG. 1.

FIG. 3 is a sketch view of a cross section of a distribution channel of the splitter in a position C of the FIG. 1.

FIG. 4 is a sketch view of a cross section of a main channel of the splitter in the FIG. 1.

FIG. 5 is a partial enlarged view of a connection part of the splitter and a guiding sleeve tube in a position A of the FIG. 1.

FIG. 6 is a partial enlarged view of a connection part of the guiding sleeve tube and a rigid sheath in a position B of the FIG. 1.

FIG. 7 is a structural sketch view of an angle adjusting mechanism of an integrated all-around adjustable flexible-rigid endoscope according to a preferred embodiment 2 of the present invention.

FIG. 8 is a structural sketch view of a steering and positioning mechanism (300) of the integrated all-around adjustable flexible-rigid endoscope according to the preferred embodiment 2 of the present invention.

FIG. 9 is a sketch view of an open groove and a limit groove shown in FIG. 8.

FIG. 10 is a structural sketch view showing that a rigid sheath and a flexible sheath cap of a flexible sheath pushing mechanism is separated according to the preferred embodiment 2 and a preferred embodiment 4 of the present invention.

FIG. 11 is a structural sketch view showing that the rigid sheath and the flexible sheath cap of the flexible sheath pushing mechanism is coupled according to the preferred embodiment 2 and the preferred embodiment 4 of the present invention.

FIG. 12 is a structural sketch view of the flexible-rigid sheath guiding mechanism according to a preferred embodiment 3 of the present invention.

FIG. 13 is a partial enlarged view of a position D in the FIG. 12.

FIG. 14 is a partial enlarged view of a position E in the FIG. 12.

FIG. 15 is a structural sketch view of the integrated all-around adjustable flexible-rigid endoscope according to the preferred embodiment 4 of the present invention.

FIG. 16 is a sketch view of the angle adjusting mechanism according to the preferred embodiment 4 of the present invention.

FIG. 17 is a sketch view of a shape of the preferred embodiment 4 of the present invention.

### DETAILED DESCRIPTION OF THE PREFERRED EMBODIMENT

Further description of the present invention is illustrated combining with the accompanying drawings, which is exemplary only and not intended to be limiting.

### Embodiment 1

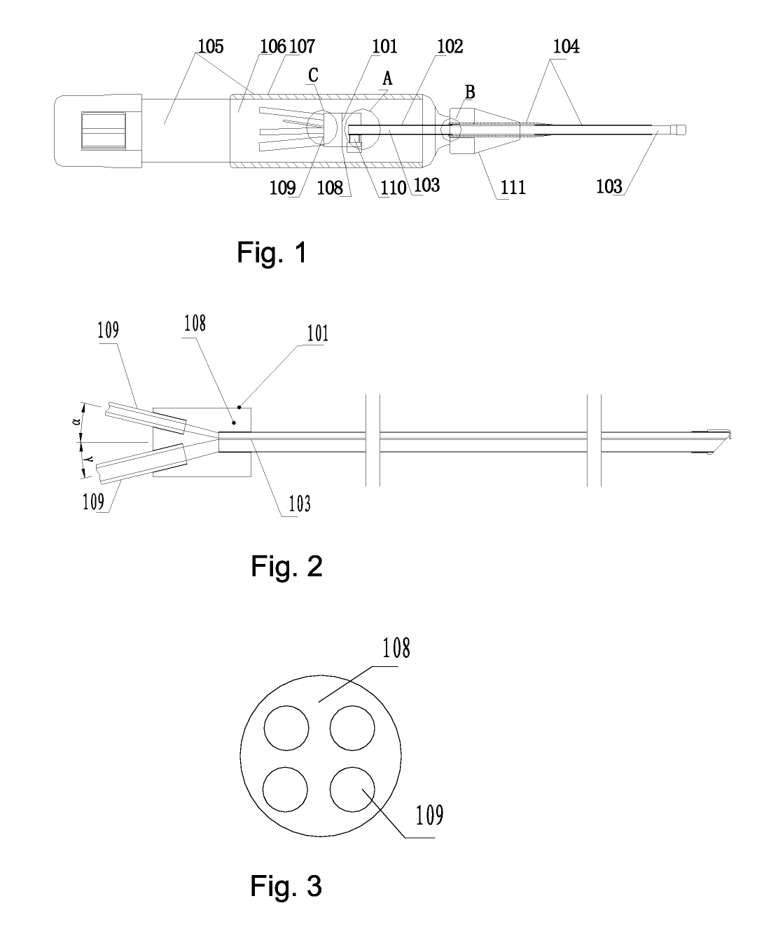

Referring to FIGS. 1-6, a flexible-rigid sheath guiding mechanism for an endoscope, according to a preferred embodiment of the present invention, comprises:

a splitter 101, a guiding sleeve tube 102, a flexible sheath 103, a rigid sheath 104 and a handle 105, wherein a first end of the guiding sleeve tube 102 is mounted on an outlet of the splitter 101, the guiding sleeve tube 102 and provided coaxially with the outlet of the splitter 101, a second end of the guiding sleeve tube 102 is inserted in the rigid sheath 104, the guiding sleeve tube 102 fits the rigid sheath 104, the handle 105 comprises a handle head 106 and a handle sleeve 107 which are fitted with each other, the splitter 101 is mounted in the handle head 106, the rigid sheath 104 is mounted on the handle sleeve 107, the flexible sheath 103 goes through the guiding sleeve tube 102 and the rigid sheath 104, and an end of the flexible sheath 103 is mounted on the outlet of the splitter 101.

The splitter 101 comprises a fixing part 108, a main channel for inserting the guiding sleeve tube 102 and the flexible sheath 103 is provided on an outlet of the fixing part 108, four distribution channels 109 opposite to the main channel are provided on the fixing part 108, wherein outer ends of the four distribution channels 109 extends to an end face of the fixing part 108, inner ends of the four distribution channels 109 extend to gather in an inner end of the main channel.

Each central axis of the four distribution channels 109 forms an included angle of 7°˜13° with an central axis of the fixing part 108.

Both the handle head 106 and the handle sleeve 107 are cylindrical structures and are matched to sleeve together.

A fastening screw 110 is provided on a side face of the main channel of the splitter 101, so as to fix the flexible sheath 103 provided on the main channel of the splitter 101.

The guiding sleeve tube 102 is made of stainless steel, and welded together with the main channel on the outlet of the splitter 101.

8A front end of the rigid sheath 104 is connected with the handle sleeve 107 via a spring cartridge 111.

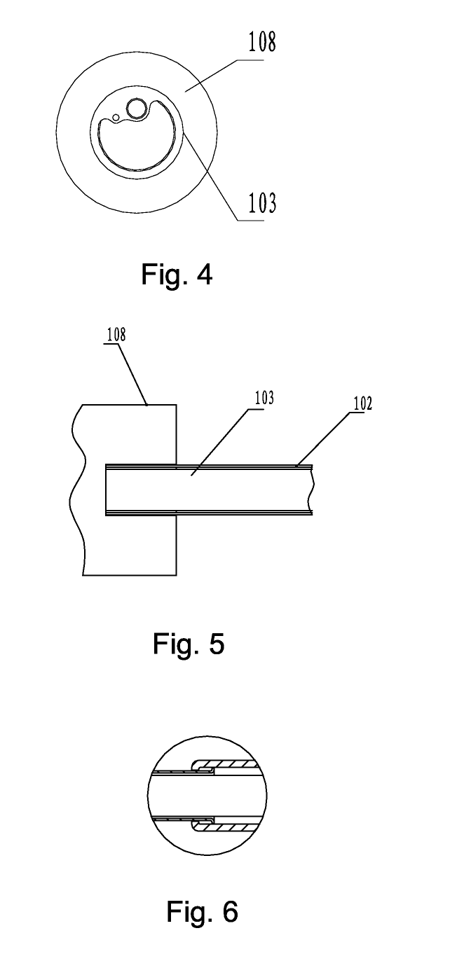

According to a preferred embodiment of the present invention, the flexible sheath 103 has three entercloses running along an axis provided therein see FIG. 4 of the drawings). The three entercloses comprise a steering control channel, an imaging and lighting sensor channel and a working channel, wherein an area of a cross section of the working channel is greater than an area of a cross section of the steering control channel or the imaging and lighting sensor channel. The cross section of the working channel is a modified cross section formed by connection of an arc and a curve. Both cross sections of the steering control channel and the imaging and lighting sensor channel are circulars. Cross sections of the three channels are provided on a circumference of the cross section of the flexible sheath.

Working principles of the flexible-rigid sheath guiding mechanism are as follows. The four distribution channels 109 are respectively connected with a device channel, an injecting channel, the imaging and lighting channel and the steering control channel. The devices, an injecting tube, the imaging and lighting sensor and the steel wire of the endoscope all enter the splitter 101 according to the distribution channels 109 respectively, so as to be gathered on another end of the four distribution channels 109, so as to enter the flexible sheath 103 in the guiding sleeve tube 102 in the distributor 101. Under the limitation of the guiding sleeve tube 102, the flexible sheath 103 extends along an axial direction of the guiding sleeve tube 102 to connect the rigid sheath 104. Since the guiding sleeve tube is a rigid tube, the guiding sleeve tube 102 sleeves the rigid sheath 104, so as to avoid direct contaction between the flexible sheath 103 and the rigid sheath 104, and that the flexible sheath 103 is capable of sleeving inside the guiding sleeve tube to process circumferential rotation and axial motion. In the actual operation, the flexible sheath is not only capable of meeting the requirements of surgical procedure to process movements of extending, retracting and rotating. Furthermore, due to the rigid characteristic of the guiding sleeve tube, the flexible sheath can be limited in a limited pathway for moving, in such a manner that the flexible sheath is not capable of deflecting and twisting.

### Embodiment 2

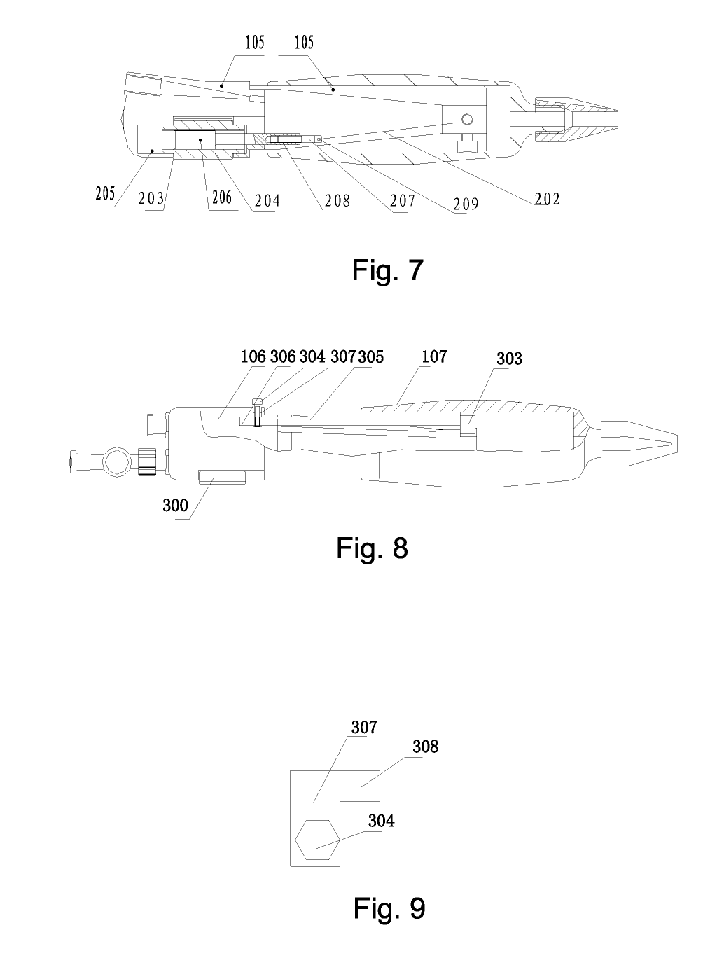

Referring to FIGS. 7˜11, a second preferred embodiment of the present invention relates to an integrated all-around adjustable flexible-rigid endoscope for the flexible-rigid sheath guiding mechanism according to the preferred embodiment 1, which not only comprises the flexible-rigid sheath guiding mechanism as recited in the preferred embodiment 1, but also comprises a bending angle adjusting mechanism 200 for the flexible sheath 103 of the endoscope, a steering and positioning mechanism 300 provided between the flexible sheath 103 and the rigid sheath 104 of the endoscope, and a flexible sheath pushing mechanism 400 provided opposite to the rigid sheath 104 for the flexible sheath 103 of the endoscope.

FIG. 7 of the drawings shows the angle adjusting mechanism 200 for the integrated all-around adjustable flexible-rigid endoscope, which comprises an angle adjusting steel wire 202, a handle 105, a nut 204 and a screw 206, wherein the screw 206 is provided on a location hole 205 of the handle 105, the nut 204 is provided on a first limit groove 203 of the handle 105, so as to cooperate with the screw 206, wherein an end of the first limit groove 203 extends to a surface of the handle 105 and has an opening, a part of an external surface of the nut 204 extends to the opening of the first limit groove 203 on the handle 105, and the angle adjusting steel wire 202 is connected with the screw 206.

wherein the screw 206 is parallel to an axis of the handle 105,

wherein the screw 206 is connected with the angle adjusting steel wire 202 via a connector 207, the connector 207 is a in a shape of a column and has screw threads on a first end thereof, a screw hole 208 is provided on the screw 206, the connector 207 is connected with thread of the screw 206, a connecting hole 209 is provided on a second end of the connector 207, and the angle adjusting steel wire 202 is mounted on the connecting hole 209,

wherein the location holes 205 are respectively provided on two axially opposite sides of the first limit groove 203,

wherein a bearing is provided between the location hole 205 and the screw 206, and

wherein a length of the first limit groove 203 is adaptive to a length of the nut 204.

The angle adjusting mechanism adopts the nut 204 and the screw 206 to overcome the disadvantages in the conventional art. The nut 204 is cooperated with the screw 206. The nut 204 is pulled to drive the screw 206 to move forwards and backwards, so as to control the angle adjusting steel wire 202 connected on the screw 206 for adjusting angle of the endoscope. Since the thread on the nut 204, which is matching with the screw 206, is designed to be threads having self-locking function, in such a manner that in the adjusting process, the screw 206 is only controlled by the nut 204, so as to further control the angle adjusting steel wire 202 connected on the screw 206. Controlling of the nut 204 is capable of conducting from an external to the angle adjusting steel wire 202, in such a manner that due to an oversize twisting moment after repeated utilization, the angle adjusting steel wire is avoid to react on the screw 206 to force the screw 206 to rebound, which leads to failure of the angle adjusting mechanism and idle of the angle adjusting steel wire 202. In the present invention, the screw 206 is an indispensable basic part. In assembly time, the nut 204 and the screw 206 can be fitted into an assembly, then the assembly is assembled into the handle 105 of the endoscope utilizing a thread.

The angle adjusting mechanism 200 of the present invention has high reliability in angle adjusting, is simple in structure and convenient in assembly and adjustment, and has advantages of accurate and steady in positioning.

Referring to FIG. 8 and FIG. 9 of the drawings, the steering and positioning mechanism 300 comprises a handle head 106, a handle sleeve 107, an eccenter 303, a driving lever 304 and a spindle 305, a location hole 306 for receiving the spindle 305 is provided in the handle head 106, a first end of the spindle 305 is connected with the eccenter 303, a second end thereof is connected with the driving lever 304, wherein the spindle 305 is provided along an axial direction of the handle head 106, an open groove 307 is provided along a circumferential direction of a surface of the handle head 106, the driving lever 304 is provided in the open groove 307, a bottom of the driving lever 304 is fixedly connected with the spindle 305, and the eccenter 303 is eccentrically provided on the spindle 305,

wherein a side face of an end of the open groove 307 has a second limit groove 308, when the driving lever 304 is received by the second limit groove 308, the eccenter 303 is compressed on an inner surface of the handle sleeve 107, and in a locked position, and

wherein a surface of the eccenter 303 is in a circular arc shape, a lower end of the driving lever 304 is in the handle head 106 and is fixedly connected with the spindle 305, and an upper end of the driving lever 304 is provided in the open groove 307 of the handle head 106.

When the steering and positioning mechanism 300 is working, the upper end of the driving lever 304 is sliding around in the open groove 307 by the operators. The sliding of the driving lever 304 in the open groove 307 drives the eccenter 303 to rotate via the spindle 305. Pull the driving lever 304 to one end to rotate the eccenter 303, in such a manner that a friction surface of the eccenter 303 is rotated from a bottom to a top, so as to be contacted with an internal friction surface of the handle head 106, so as to increase frictional force between the eccenter 303 and the internal friction surface of the handle head 106 to achieve steering and positioning. In actual operation of the endoscope, the handle head 106 and the handle sleeve are cooperated to work. After explore lesions, when the physician want to do further treatment according to the lesions explored, the endoscope is required for accurately positioning and targeting on the lesions. At this moment, just pull the driving lever 304, in such a manner that the driving lever 304 slides in the open groove 307 on the external surface of the handle head 106, which drives the spindle 305 to do circumferential rotation to further drive the eccenter 303 to do circumferential rotation, in such a manner that the friction surface of the eccenter 303 is converted from the bottom to the top, in such a manner that the friction surface of the eccenter 303 is contacted with the internal surface of the handle head 106 to increase the frictional force therebetween, in such a manner that the handle head 106 and the handle sleeve 107 achieve limited connection.

When the flexible sheath of the endoscope is inserted in human body, and processes rotating operation, the steering and positioning mechanism begins to process positioning.

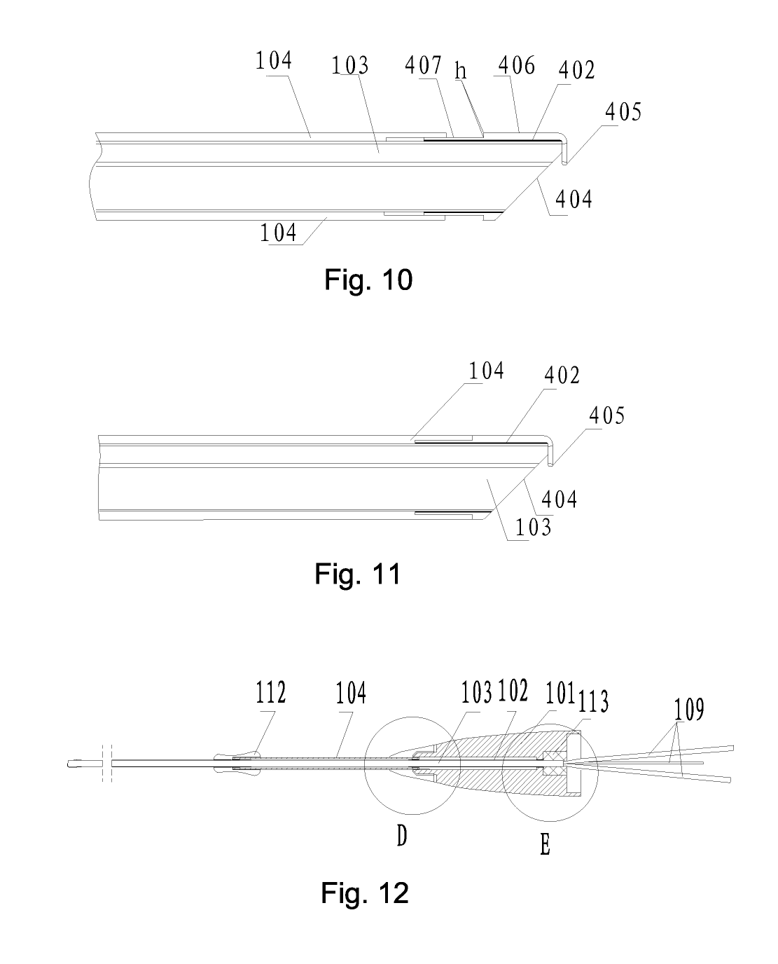

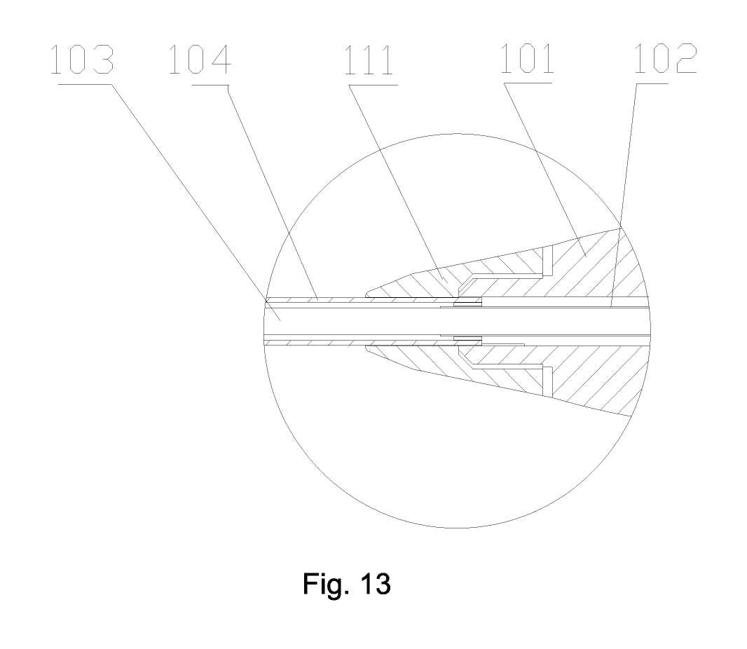

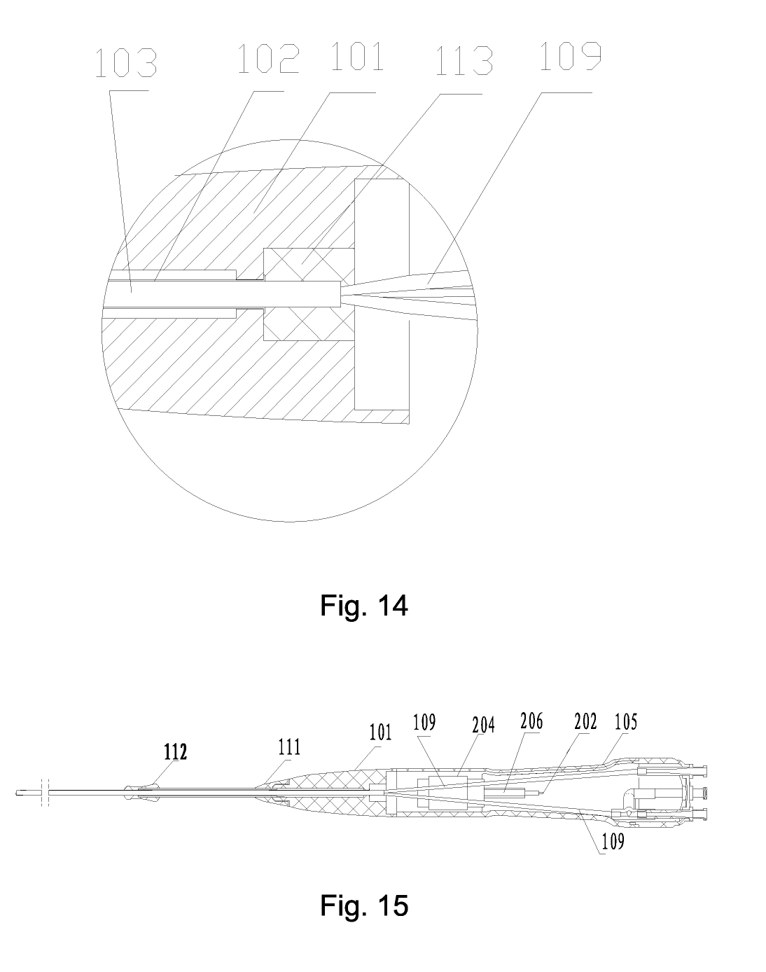

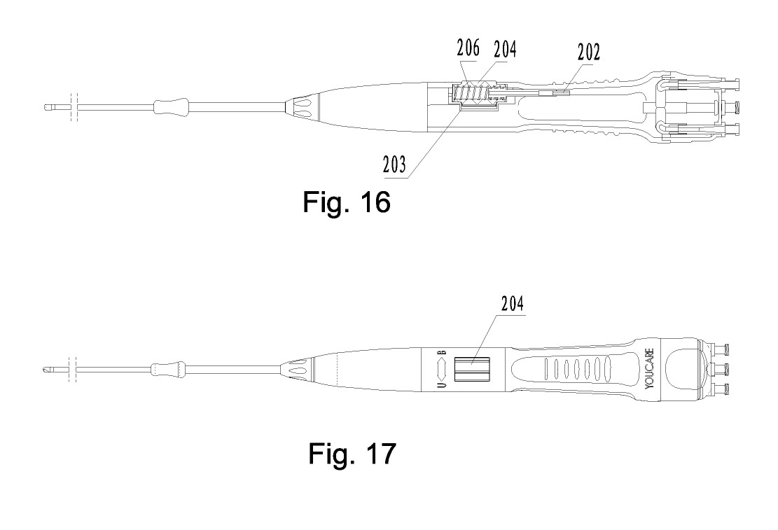

Referring to FIGS. 10 and 11, the flexible sheath pushing mechanism 400 integrated all-around adjustable flexible-rigid endoscope, comprises the flexible sheath 103, a flexible sheath cap 402 and the rigid sheath 104, wherein the flexible sheath cap 402 is in a shape of a circular tube, an external surface of the flexible sheath cap 402 has two stepped faces with different diameters, wherein an external surface of a first stepped face 406 with a greater diameter is in a streamlined shape along the axial direction, an external surface of a second stepped face 407 with a smaller diameter is a circular surface which is linear along the axial direction and capable of matching with an inner bore of the rigid sheath 104, an end face 404 with a greater diameter of the flexible sheath cap 402 is an inclined plane, and a barrier chip 405 for fixing steel wire is provided on the end face 404, an inner bore of the flexible sheath cap 402 is sleeved on an external surface of a head of the flexible sheath 103, and is mounted thereon, an end of the steel wire in the flexible sheath 103 is fixedly connected with the barrier chip 405; the rigid sheath 104 is sleeved on an external of the flexible sheath 103, an external end face of the rigid sheath 104 is capable of matching with a stepped end face of the flexible sheath cap 402,

wherein a step height h between the first stepped face and the second step face of the flexible sheath cap 402 is equal to a thickness of the rigid sheath 104 matched therewith,

wherein the barrier chip 405 and the flexible sheath cap 402 forms an integrated structure, the barrier chip 405 is provided on a farthest portion of the inclined end face 404,

wherein the barrier chip 405 is provided on a position corresponding to a steering control channel on a cross section of the flexible sheath cap 402, and

wherein when the rigid sheath 104 is cooperated with the flexible sheath cap 402, an external diameter of the rigid sheath 104 is equal to a diameter of a smaller end of the stepped face 406 with a greater diameter of the flexible sheath cap 402.

The present invention according to the preferred embodiment further provides a flexible sheath for the endoscope, wherein a flexible sheath cap 402 is provided on a head of the flexible sheath 103, and the head of the flexible sheath 103 is inserted in a hole of the flexible sheath cap 402.

In the preferred embodiment, the flexible 402 is sleeved in the flexible sheath cap 103, so as to match with the opening of the flexible sheath. When the endoscope is inserted deeply in the human body, the barrier chip 405 of the flexible sheath cap 402 prevents the opening of the flexible sheath separating from flexible sheath and forms a whole. Smooth outer ring surface of the flexible sheath cap 402 ensures that the head of the flexible sheath is not easy to stab and wound human tissue during the inserting operation, which increases safety of utilizing the endoscope in the operation. During the rotating or retracting operation of the endoscope, since the thickness of the flexible sheath cap 402 is greater than the gap between the flexible sheath 103 and the rigid sheath 104, the flexible sheath is avoided to shrink into the rigid sheath 403. The clinical significance of the structure lies in being capable of solving technical problems caused by lacking the flexible sheath cap in the flexible sheath, e.g., during the surgery, the flexible sheath runs along or punctures nature orifice of human body, the exploration is difficult to process because material of the flexible sheath lacks puncture hardness; and the flexible sheath is easy to contracted into the rigid sheath, which causes a result that co-movements of the flexible sheath and the rigid sheath not strong and thus are easy to separate.

### Embodiment 3

Referring to FIGS. 12˜14 of the drawings, the preferred embodiment relates to a second flexible-rigid sheath guiding mechanism for an endoscope, which comprises a splitter 101, a guiding sleeve tube 102, a flexible sheath 103, a rigid sheath 104 and a handle 105, wherein a first end of the guiding sleeve tube 102 is mounted on a main channel an outlet of the splitter 101, the guiding sleeve tube 102 and provided coaxially with the main channel outlet of the splitter 101, a second end of the guiding sleeve tube 102 is inserted in the rigid sheath 104, the guiding sleeve tube 102 fits the rigid sheath 104, wherein the splitter 101 is a part of a head of the handle and is mounted on the handle 105, a circular main channel is provided on a front end of the splitter 101, wherein the guiding sleeve tube 102 is provided on the main channel, a gap is provided between an external surface of the guiding sleeve tube 102 and an internal surface of the main channel, a rear end of the rigid sheath is sleeved on an external of the guiding sleeve tube 102 and is inserted into the gap, three distribution channels 109 are provided on an end opposite to the main channel on the splitter 101,

wherein the three distribution channels 109 respectively comprise a apparatus catheter, a stainless steel capillary, and an imaging and lighting sensor catheter, wherein a chamber is provided on a root of the splitter 101, internal ends of the apparatus catheter, the stainless steel capillary, the imaging and lighting sensor catheter, the guiding sleeve tube 102 and the flexible sheath 103 extend to gather in the internal end of the main channel,

wherein each central axis of the three distribution channel chambers 109 forms an included angle of 7°˜13° with an central axis of the main channel fixing part 108, and

wherein the flexible sheath 103 has three entercloses running along an axis provided therein, the three entercloses are respectively communicated with the apparatus catheter, the stainless steel capillary and the imaging and lighting sensor catheter in the three distribution channels 109 of the splitter 101.

An external surface of the rigid sheath has an annular ligament 112 mounted thereon for holding.

### Embodiment 4

Referring to FIGS. 12˜17, an integrated all-around adjustable flexible-rigid endoscope utilizing the flexible-rigid sheath guiding mechanism not only comprises the flexible-rigid sheath guiding mechanism according to the preferred embodiment 3, but also comprises a bending angle adjusting mechanism 200 for the flexible sheath 103 of the endoscope, a steering and positioning mechanism 300 provided between the flexible sheath 103 and the rigid sheath 104 of the endoscope, and a flexible sheath pushing mechanism 400 provided opposite to the rigid sheath 104 for the flexible sheath 103 of the endoscope.

The structure and working principle of the angle adjusting mechanism in the preferred embodiment 4 are basically the same with thereof in the preferred embodiment 2. The slight difference lies in that in the preferred embodiment 4, the steel wire passes a central through hole of the screw 206 which is hollow and is fixed on an external end of the screw 206.

In the preferred embodiment 4, structure of the pushing mechanism 400 is completely the same with thereof in the preferred embodiment 2, and the accompanying drawings are the same as well and thus are omitted here.

Fundamental difference between the preferred embodiment 4 and the preferred embodiment 2 lies in the steering and positioning mechanism 300 comprises a collet 111 which is matching with thread on a front end of the main channel of the splitter 101, the collet 111 provided on the front end of the main channel is capable of processing elastic contraction, when the collet 111 is tightened, the front end of the main channel is contracted to grip the rigid sheath 104, in such a manner that the rigid sheath 104 is fixedly connected with the splitter 101.

The difference between the preferred embodiments 3, 4 and the preferred embodiments 1, 2 lies in that in the preferred embodiments 3 and 4, the handle 105 is a whole, and in the preferred embodiments 1 and 2, the handle comprises the handle head 106 and the handle sleeve 107. However, the preferred embodiments 3 and 4 further comprises an annular ligament mounted on the external of the rigid sheath, which is, from the functional point of view, equivalent to the handle sleeve 107 in the preferred embodiments 1 and 2.

Furthermore, from the appearances, the splitters in the preferred embodiments 3 and 4 are different from the preferred embodiment 1 and 2. In the preferred embodiment 3 and 4, the splitter is mounted on the front end of the handle, and forms a whole with the handle 105, but in the preferred embodiments 1 and 2, the splitter 101 is mounted in the handle head 106. However, the splitters in the preferred embodiments 3 and 4 are essentially the same with thereof in the preferred embodiments 1 and 2, because the handle 105 is functionally equivalent to the handle head 106 in the preferred embodiments 1 and 2.

Working principles of the steering and positioning mechanism are as follows. One hand holds the handle 105, the other hand holds the annular ligament 112 on the external surface of the rigid sheath 104, so as to process rotating, expanding and contracting, so as to adjust relative rotating angle, expanding or contracting distance in the axial direction. When the adjustment is in position, the collet 111 of the steering and positioning mechanism is fastened, so as to fix the rigid sheath 104 on the splitter 101, so as to lock the relative position between the rigid sheath and the flexible sheath. Other parts and connectors of the endoscope are conventional arts, and are not illustrated in detail here.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1. A flexible-rigid sheath guiding mechanism for an endoscope, comprising: a splitter (101), a guiding sleeve tube (102), a flexible sheath (103), a rigid sheath (104) and a handle (105), wherein a first end of the guiding sleeve tube (102) is mounted on an outlet of the splitter (101), the guiding sleeve tube (102) and provided coaxially with the outlet of the splitter (101), a second end of the guiding sleeve tube (102) is inserted in the rigid sheath (104), the guiding sleeve tube (102) fits the rigid sheath (104), the handle (105) comprises a handle head (106) and a handle sleeve (107) which are fitted with each other, the splitter (101) is mounted in the handle head (106), the rigid sheath (104) is mounted on the handle sleeve (107), the flexible sheath (103) goes through the guiding sleeve tube (102) and the rigid sheath (104), and an end of the flexible sheath (103) is mounted on the outlet of the splitter (101).

2. The flexible-rigid sheath guiding mechanism for the endoscope, as recited in claim 1, wherein the splitter (101) comprises a fixing part (108), a main channel for inserting the guiding sleeve tube (102) and the flexible sheath (103) is provided on an outlet of the fixing part (108), four distribution channels (109) opposite to the main channel are provided on the fixing part (108), wherein outer ends of the four distribution channels (109) extends to an end face of the fixing part (108), inner ends of the four distribution channels (109) extend to gather in an inner end of the main channel.

3. The flexible-rigid sheath guiding mechanism for the endoscope, as recited in claim 2, wherein each central axis of the four distribution channels (109) forms an included angle of 7°˜13° with an central axis of the fixing part (108).

4. The flexible-rigid sheath guiding mechanism for the endoscope, as recited in claim 2, wherein a fastening screw (110) is provided on a side face of the main channel of the splitter (101), so as to fix the flexible sheath (103) provided on the main channel of the splitter (101).

5. The flexible-rigid sheath guiding mechanism for the endoscope, as recited in claim 1, wherein the guiding sleeve tube (102) is made of stainless steel, and welded together with the main channel on the outlet of the splitter (101).

6. The flexible-rigid sheath guiding mechanism for the endoscope, as recited in claim 1, wherein the flexible sheath (103) has three entercloses running along an axis provided therein.

7. The flexible-rigid sheath guiding mechanism for the endoscope, as recited in claim 1, wherein both the handle head (106) and the handle sleeve (107) are cylindrical structures and are matched to sleeve together.

8. The flexible-rigid sheath guiding mechanism for the endoscope, as recited in claim 1, wherein a front end of the rigid sheath (104) is connected with the handle sleeve (107) via a spring cartridge (111).

9. A flexible-rigid sheath guiding mechanism and integrated all-around adjustable flexible-rigid endoscope thereof, as recited in claim 1, further comprising a bending angle adjusting mechanism (200) for the flexible sheath (103) of the endoscope, a steering and positioning mechanism (300) provided between the flexible sheath (103) and the rigid sheath (104) of the endoscope, and a flexible sheath pushing mechanism (400) provided opposite to the rigid sheath (104) for the flexible sheath (103) of the endoscope.

10. The integrated all-around adjustable flexible-rigid endoscope, as recited in claim 9, wherein the angle adjusting mechanism (200) comprises an angle adjusting steel wire (202), a handle (105), a nut (204) and a screw (206), wherein the screw (206) is provided on a location hole (205) of the handle (105), the nut (204) is provided on a first limit groove (203) of the handle (105), so as to cooperate with the screw (206), wherein an end of the first limit groove (203) extends to a surface of the handle (105) and has an opening, a part of an external surface of the nut (204) extends to the opening of the first limit groove (203) on the handle (105), and the angle adjusting steel wire (202) is connected with the screw (206).

11. The integrated all-around adjustable flexible-rigid endoscope, as recited in claim 10, wherein the screw (206) is parallel to an axis of the handle (105).

12. The integrated all-around adjustable flexible-rigid endoscope, as recited in claim 10, wherein the screw (206) is connected with the angle adjusting steel wire (202) via a connector (207), the connector (207) is a in a shape of a column and has screw threads on a first end thereof, a screw hole (208) is provided on the screw (206), the connector (207) is connected with thread of the screw (206), a connecting hole (209) is provided on a second end of the connector (207), and the angle adjusting steel wire (202) is mounted on the connecting hole (209).

13. The integrated all-around adjustable flexible-rigid endoscope, as recited in claim 10, wherein the location holes (205) are respectively provided on two axially opposite sides of the first limit groove (203).

14. The integrated all-around adjustable flexible-rigid endoscope, as recited in claim 10, wherein a bearing is provided between the location hole (205) and the screw (206).

15. The integrated all-around adjustable flexible-rigid endoscope, as recited in claim 10, wherein a length of the first limit groove (203) is adaptive to a length of the nut (204).

16. The integrated all-around adjustable flexible-rigid endoscope, as recited in claim 9, wherein the steering and positioning mechanism (300) comprises a handle head (106), a handle sleeve (107), an eccenter (303), a driving lever (304) and a spindle (305), a location hole (306) for receiving the spindle (305) is provided in the handle head (106), a first end of the spindle (305) is connected with the eccenter (303), a second end thereof is connected with the driving lever (304), wherein the spindle (305) is provided along an axial direction of the handle head (106), an open groove (307) is provided along a circumferential direction of a surface of the handle head (106), the driving lever (304) is provided in the open groove (307), a bottom of the driving lever (304) is fixedly connected with the spindle (305), and the eccenter (303) is eccentrically provided on the spindle (305).

17. The integrated all-around adjustable flexible-rigid endoscope, as recited in claim 16, wherein a side face of an end of the open groove (307) has a second limit groove (308), when the driving lever (304) is received by the second limit groove (308), the eccenter (303) is compressed on an inner surface of the handle sleeve (107), and in a locked position.

18. The integrated all-around adjustable flexible-rigid endoscope, as recited in claim 16, wherein a surface of the eccenter (303) is in a circular arc shape.

19. The integrated all-around adjustable flexible-rigid endoscope, as recited in claim 9, wherein the flexible sheath pushing mechanism (400) comprises the flexible sheath (103), a flexible sheath cap (402) and the rigid sheath (104), wherein the flexible sheath cap (402) is in a shape of a circular tube, an external surface of the flexible sheath cap (402) has two stepped faces with different diameters, wherein an external surface of a first stepped face (406) with a greater diameter is in a streamlined shape along the axial direction, an external surface of a second stepped face (407) with a smaller diameter is a circular surface which is linear along the axial direction and capable of matching with an inner bore of the rigid sheath (104), an end face (404) with a greater diameter of the flexible sheath cap (402) is an inclined plane, and a barrier chip (405) for fixing steel wire is provided on the end face (404), an inner bore of the flexible sheath cap (402) is sleeved on an external surface of a head of the flexible sheath (103), and is mounted thereon, an end of the steel wire in the flexible sheath (103) is fixedly connected with the barrier chip (405); the rigid sheath (104) is sleeved on an external of the flexible sheath (103), an external end face of the rigid sheath (104) is capable of matching with a stepped end face of the flexible sheath cap (402).

20. The integrated all-around adjustable flexible-rigid endoscope, as recited in claim 19, wherein a step height h between the first stepped face and the second step face of the flexible sheath cap (402) is equal to a thickness of the rigid sheath (104) matched therewith.

21. The integrated all-around adjustable flexible-rigid endoscope, as recited in claim 19, wherein the barrier chip (405) and the flexible sheath cap (402) forms an integrated structure, the barrier chip (405) is provided on a farthest portion of the inclined end face (404).

22. The integrated all-around adjustable flexible-rigid endoscope, as recited in claim 19, wherein the barrier chip (405) is provided on a position corresponding to a steering control channel on a cross section of the flexible sheath cap (402).

23. A flexible-rigid sheath guiding mechanism for an endoscope, comprising: a splitter (101), a guiding sleeve tube (102), a flexible sheath (103), a rigid sheath (104) and a handle (105), wherein a first end of the guiding sleeve tube (102) is mounted on a main channel of the splitter (101), the guiding sleeve tube (102) and provided coaxially with the main channel of the splitter (101), a second end of the guiding sleeve tube (102) is inserted in the rigid sheath (104), the guiding sleeve tube (102) fits the rigid sheath (104), the splitter (101) is mounted on the handle (105), the flexible sheath (103) goes through the guiding sleeve tube (102) and the rigid sheath (104), and an end of the flexible sheath (103) is mounted on the main channel of the splitter (101), an external surface of the rigid sheath (104) is fixed and has an annular ligament (112) for holding.

24. The flexible-rigid sheath guiding mechanism for the endoscope, as recited in claim 23, wherein the splitter (101) is a part of a head of the handle, a circular main channel is provided on a front end of the splitter (101), wherein the guiding sleeve tube (102) is provided on the main channel, a gap is provided between an external surface of the guiding sleeve tube (102) and an internal surface of the main channel, a rear end of the rigid sheath is sleeved on an external of the guiding sleeve tube (102) and is inserted into the gap, three distribution channels (109) are provided on an end opposite to the main channel on the splitter (101), and inner ends of the three distribution channels (109) extend to be gathered in an inner end of the main channel.

25. The flexible-rigid sheath guiding mechanism for the endoscope, as recited in claim 24, wherein the three distribution channels (109) respectively comprise a apparatus catheter, a stainless steel capillary, and an imaging and lighting sensor catheter, wherein a chamber is provided on a root of the splitter (101), internal ends of the apparatus catheter, the stainless steel capillary, the imaging and lighting sensor catheter, the guiding sleeve tube (102) and the flexible sheath (103) extend to gather in the internal end of the main channel.

26. The flexible-rigid sheath guiding mechanism for the endoscope, as recited in claim 24, wherein each central axis of the three distribution channels (109) forms an included angle of 7°˜13° with an central axis of the main channel.

27. The flexible-rigid sheath guiding mechanism for the endoscope, as recited in claim 24, wherein the flexible sheath (103) has three entercloses running along an axis provided therein, the three entercloses are respectively communicated with the three distribution channels (109) of the splitter (101).

28. The flexible-rigid sheath guiding mechanism for the endoscope, as recited in claim 23, further comprising a bending angle adjusting mechanism (200) for the flexible sheath (103) of the endoscope, a steering and positioning mechanism (300) provided between the flexible sheath (103) and the rigid sheath (104) of the endoscope, and a flexible sheath pushing mechanism (400) provided opposite to the rigid sheath (104) for the flexible sheath (103) of the endoscope.

29. The flexible-rigid sheath guiding mechanism for the endoscope, as recited in claim 28, wherein the steering and positioning mechanism (300) comprises a collet (111) which is matching with thread on a front end of the main channel of the splitter (101), the collet (111) provided on the front end of the main channel is capable of processing elastic contraction, when the collet (111) is tightened, the front end of the main channel is contracted to grip the rigid sheath (104), in such a manner that the rigid sheath (104) is fixedly connected with the splitter (101).

30. The flexible-rigid sheath guiding mechanism for the endoscope, as recited in claim 28, wherein the angle adjusting mechanism (200) comprises an angle adjusting steel wire (202), a handle (105), a nut (204) and a screw (206), wherein the screw (206) is provided on a location hole (205) of the handle (105), the nut (204) is provided on a first limit groove (203) of the handle (105), so as to cooperate with the screw (206), wherein an end of the first limit groove (203) extends to a surface of the handle (105) and has an opening, a part of an external surface of the nut (204) extends to the opening of the first limit groove (203) on the handle (105), and the angle adjusting steel wire (202) is connected with the screw (206).

31. The flexible-rigid sheath guiding mechanism for the endoscope, as recited in claim 30, wherein the screw (206) is parallel to an axis of the handle (105).

32. The flexible-rigid sheath guiding mechanism for the endoscope, as recited in claim 30, wherein the location holes (205) are respectively provided on two axially opposite sides of the first limit groove (203).

33. The integrated all-around adjustable flexible-rigid endoscope, as recited in claim 30, wherein a bearing is provided between the location hole (205) and the screw (206).

34. The integrated all-around adjustable flexible-rigid endoscope, as recited in claim 30, wherein a length of the first limit groove (203) is adaptive to a length of the nut (204).

35. The integrated all-around adjustable flexible-rigid endoscope, as recited in claim 28, wherein the flexible sheath pushing mechanism (400) comprises the flexible sheath (103), a flexible sheath cap (402) and the rigid sheath (104), wherein the flexible sheath cap (402) is in a shape of a circular tube, an external surface of the flexible sheath cap (402) has two stepped faces with different diameters, wherein an external surface of a first stepped face (406) with a greater diameter is in a streamlined shape along the axial direction, an external surface of a second stepped face (407) with a smaller diameter is a circular surface which is linear along the axial direction and capable of matching with an inner bore of the rigid sheath (104), an end face (404) with a greater diameter of the flexible sheath cap (402) is an inclined plane, and a barrier chip (405) for fixing steel wire is provided on the end face (404), an inner bore of the flexible sheath cap (402) is sleeved on an external surface of a head of the flexible sheath (103), and is mounted thereon, an end of the steel wire in the flexible sheath (103) is fixedly connected with the barrier chip (405); the rigid sheath (104) is sleeved on an external of the flexible sheath (103), an external end face of the rigid sheath (104) is capable of matching with a stepped end face of the flexible sheath cap (402).

36. The integrated all-around adjustable flexible-rigid endoscope, as recited in claim 35, wherein a step height h between the first stepped face and the second step face of the flexible sheath cap (402) is equal to a thickness of the rigid sheath (104) matched therewith.

37. The integrated all-around adjustable flexible-rigid endoscope, as recited in claim 35, wherein the barrier chip (405) and the flexible sheath cap (402) forms an integrated structure, the barrier chip (405) is provided on a farthest portion of the inclined end face (404).

38. The integrated all-around adjustable flexible-rigid endoscope, as recited in claim 35, wherein the barrier chip (405) is provided on a position corresponding to a steering control channel on a cross section of the flexible sheath cap (402).
